(19) United States
(12) Patent Application Publication
(10) Pub. No.: US 2026/0137612 A1
(43) Pub. Date: May 21, 2026

(54) COMPOSITION FOR RELIEVING SKIN ITCHING OR SKIN SOOTHING

(71) Applicant: LG H&H CO., LTD., Seoul (KR)

(72) Inventors: Seongsu KANG, Seoul (KR); Seunghyun JUN, Seoul (KR); Younjung LEE, Seoul (KR); Euitaek JEONG, Seoul (KR)

(73) Assignee: LG H&H CO., LTD., Seoul (KR)

(21) Appl. No.: 19/390,942

(22) Filed: Nov. 17, 2025

(30) Foreign Application Priority Data
Nov. 18, 2024 (KR) 10-2024-0164239

(51) Int. Cl. A61K 8/63; A61K 8/60; A61K 31/575; A61K 31/7048; A61P 17/04; A61P 25/02; A61P 29/00; A61P 37/08; A61Q 19/00

(52) U.S. Cl. CPC A61K 8/63; A61K 8/602; A61K 31/575; A61K 31/7048; A61P 17/04; A61P 25/02; A61P 29/00; A61P 37/08; A61Q 19/00; A61Q 19/007

(57) ABSTRACT

The present disclosure relates to a composition for skin improvement comprising beta-sitosterol and betulin. It was confirmed that the composition has excellent effects of skin-soothing and relieving skin irritation by reducing skin nerve stimulation-inducing factors, increasing the expression of moisturizing-related factors, increasing skin absorption, improving skin permeation, and inhibiting mast cell activity, and thus can be effectively used in cosmetics, quasi-drugs, etc. for skin improvement.

### TECHNICAL FIELD

The present disclosure relates to a composition for skin improvement including beta-sitosterol and betulin. The composition can relieve skin itching, and has a skin soothing effect and significantly improves skin permeability.

### BACKGROUND ART

The skin is the primary barrier of the human body, which functions to protect the organs of the body from external environmental stimuli such as changes in temperature and humidity, UV rays and pollutants. At the same time, the skin reacts sensitively to external stimuli, acting as a type of sensor. Skin sensitivity is influenced by genetic factors, but is also significantly influenced by external factors. For example, environmental pollution, UV rays, dry air, chemical substances, and stress can trigger skin sensitivity, and the use of inappropriate skincare products can also worsen it. Therefore, people with sensitive skin are advised to use products with appropriate soothing or irritation-reducing effects to protect their skin.

Excessive skin hypersensitivity, irritation, and itching are caused by various physiological mechanisms. First, damage to the skin barrier mediates various inflammatory responses, leading to various pathological phenomena such as irritation, itching, etc. (James Del Rosso, J Clin Aesthet Dermatol. 2016). The most prominent problem in atopic dermatitis and psoriasis is skin barrier damage, and its restoration has been shown to alleviate skin inflammation and irritation. Similarly, skin humidity and dryness are also associated with skin sensitivity. Dry environments increase damage to the skin barrier and the penetration of external substances (M Denda, J Invest Dermatol. 1998), leading to increased inflammatory responses (Y. Ashida, Br J Dermatol. 2003). Therefore, skin moisture content or moisture-maintaining ability is an important factor in managing skin sensitivity.

Recent studies have shown that the activity of the transient receptor potential vanilloid 1 (TRPV1) receptor in skin cells and nerve cells within skin tissue significantly influences skin inflammation and sensitivity (Michelle D. Bagood, Int. J. Mol. Sci. 2021). The TRPV1 receptor is an ion channel receptor that regulates neurotransmission in response to temperature and chemical stimuli. It induces neurotransmission signals by allowing calcium cations to enter the cell in response to temperature, pH, and chemical stimuli. In the skin, TRPV1 performs physiological functions such as temperature regulation, pain detection, and heat perception, etc. It has been shown that people with sensitive skin have overexpressed TRPV1 (Adriana Ehnis-Pérez, J Cosmet Dermatol. 2016), and treatment with TRPV1 agonists has been shown to alleviate such skin irritation (Thomas Kueper, Exp Dermatol. 2010).

Mast cells within skin tissue produce various inflammatory mediators, such as histamine, etc., in response to external stimuli, inducing allergic reactions and itching. It is known that the inhibition of such reaction suppresses skin inflammation and itching (Tiphaine Voisin, Immunity. 2019).

Although various cosmetic ingredients and skincare ingredients for external use have been developed, compositions exhibiting all of the above effects or showing a satisfactory level of efficacy are limited.

Accordingly, the inventors of the present disclosure made extensive efforts to develop substances that could relieve itching and improve skin conditions, and discovered that a combination of betulin, sitosterol, and troxerutin effectively relieved and soothed itching.

The outer layer of the skin consists of 10 to 15 corneocyte layers with a thickness of 10 to 45 μm. These are organized into a form known as mortar and brick structures. The brick structure consists of corneocytes rich in keratin, and the spaces between corneocytes are filled with ceramides, fatty acids, waxes, etc., acting as mortars. The structure prevents the loss of internal moisture and the invasion from the outside. Therefore, although external substances may have difficulty penetrating the skin layer, low-molecular-weight components having 500 Da or less can be delivered into the skin by diffusion (Exp. Dermatol., 2000, 9, 165-9.). Alternatively, they can be delivered through sweat glands, pores of the skin, sebaceous glands, etc. without penetrating the corneocyte layers.

For this reason, active research is underway to develop methods that can penetrate the skin and deliver substances evenly throughout the skin, regardless of molecular size or properties.

Under the above circumstances, the inventors of the present disclosure discovered that beta-sitosterol, betulin, and troxerutin exhibit excellent skin improvement effects, specifically excellent effects in relieving itching, skin soothing, and enhancing skin permeation, thereby completing the present disclosure.

### DISCLOSURE

### Technical Problem

It is one object of the present disclosure to provide a composition for skin improvement, comprising beta-sitosterol and betulin as active ingredients.

The composition has the effects of significantly relieving skin itching, providing skin soothing and improving skin permeability.

It is another object of the present disclosure to provide a cosmetic composition for skin improvement, comprising beta-sitosterol and betulin as active ingredients.

It is still another object of the present disclosure to provide a quasi-drug composition for skin improvement, comprising beta-sitosterol and betulin as active ingredients.

### Technical Solution

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

Additionally, those of ordinary skill in the art may be able to recognize or confirm, using only conventional experimentation, many equivalents to the particular aspects of the invention described herein. Furthermore, it is also intended that these equivalents be included in the present disclosure.

In order to achieve the objects above, one aspect of the present disclosure provides a composition for skin improvement, comprising beta-sitosterol and betulin as active ingredients.

As used herein, the term “beta-sitosterol” has a chemical structure similar to that of cholesterol and is one of plant sterols. Beta-sitosterol has been used as a drug for treating skin burns and wounds. The “beta-sitosterol” of the present disclosure may be used interchangeably with the term “sitosterol.”

The beta-sitosterol of the present disclosure may be those isolated or separated from an extract obtained from a plant containing beta-sitosterol as an active ingredient, or a fraction thereof, but there is no limitation on the method for obtaining the same.

In the present disclosure, the beta-sitosterol may be contained in an amount of 0.00001 to 3 wt % (w/w) based on the total weight of the composition. Specifically, it may be contained in an amount of 0.00001 to 3%, 0.001 to 2%, 0.01 to 1%, 0.05 to 1%, or 0.1 to 1%, but is not limited thereto.

As used herein, the term “betulin” refers to a triterpene compound obtained from the bark or sap of birch trees. It is known to have anticancer, sedative, and antiaging effects. The betulin of the present disclosure may be those isolated or separated from an extract obtained from a plant containing betulin as an active ingredient, or a fraction thereof, but there is no limitation on the method for obtaining the same.

In the present disclosure, the betulin may be contained in an amount of 0.00001 to 3 wt % (w/w) based on the total weight of the composition. Specifically, it may be contained in an amount of 0.00001 to 3%, 0.001 to 2%, 0.01 to 1%, 0.05 to 1%, or 0.1 to 1%, but is not limited thereto.

Additionally, in the present disclosure, the beta-sitosterol and betulin can be mixed in a weight ratio of 10:1 to 1:10, specifically 7:1 to 1:7, and more specifically 5:1 to 1:5 or 3:1 to 1:3, but is not limited thereto.

As used herein, the term “skin permeation” refers to the ability to penetrate a biological membrane and permeate into the skin. The biological membrane may refer to, but is not limited to, cell permeation, skin absorption, and/or mucosal permeability. In the present disclosure, the “skin permeability” refers to the measurement of the skin permeation rate of medications (in the form of ointments, patches, sprays, etc.) and cosmetics applied to the skin.

As used herein, the term “improvement” refers to all activities that at least reduce the parameters associated with alleviation or treatment of conditions, e.g., the degree of symptoms, and may include any one or more among skin moisturization, skin soothing, skin regeneration, improvement of skin inflammation, skin protection from external stimuli, soothing of sensitive skin, improvement of sensitive skin, alleviation of erythema, relief of itching, enhancement of skin barrier function, alleviation of skin irritation, improvement of skin permeation, and alleviation of skin pain, and alleviation of skin pain.

In one embodiment of the present disclosure, it was confirmed that the combination of beta-sitosterol and betulin exhibited skin improvement effects, specifically, the effects of significantly improving neurogenic inflammation, skin soothing, relieving itching, enhancing moisturization, anti-allergy, and skin permeation, compared to betulin alone, and moreover, can improve stability in solvents.

The composition may be for improving skin permeation, but is not limited thereto.

In the present disclosure, the composition including beta-sitosterol and betulin has the effects of anti-inflammation, skin soothing, relieving itchiness, enhancing moisturization, anti-allergy, and alleviating skin irritation.

As used herein, the term “inflammation” is a kind of in vivo response to damage or infection of a specific tissue, and the main mediator is immune cells. The purpose of such inflammation is to suppress tissue damage as much as possible, remove infectious agents, and regenerate tissue. In the present disclosure, inflammation may refer to neurogenic inflammation or inflammatory skin diseases, but is not limited thereto.

As used herein, the term “neurogenic inflammation, neurogenic inflammatory disease or neurogenic inflammatory skin disease” refers to an inflammation limited to the peripheral nervous system (PNS) and central nervous system (CNS), or a skin disease accompanying the same or arising therefrom. The neurogenic inflammation is triggered by nerve activation and causes pain, itching, rash, edema, swelling, etc. of the skin. It is known to mediate a long-term inflammatory response by inducing diffused lesions in a short period of time. The neurogenic inflammatory disease or neurogenic inflammatory skin disease may include, but is not limited to, atopic dermatitis, eczema, psoriasis, rash, and prurigo nodularis (PN). Additionally, the neurogenic inflammation has been recognized as a major cause of sensitive skin.

As used herein, the term “transient receptor potential vanilloid 1 (TRPV1) receptor or cutaneous neurogenic irritation-induced receptor TRPV1” is a receptor located in large numbers on skin cells and nerve cells within the skin, and plays a role in recognizing external stimuli or pain and inducing an inflammatory response in surrounding tissues. When TRPV1 is excessively activated, the threshold for itching is reduced, which means that itchy sensation (itching) increases, potentially leading to sensitive skin.

Accordingly, sensitive skin can be improved by suppressing TRPV1. In one embodiment of the present disclosure, it was confirmed that the composition of the present disclosure exhibited an improvement effect on sensitive skin by suppressing TRPV1.

Specifically, in one embodiment of the present disclosure, it was confirmed that the combination of beta-sitosterol and betulin exhibited a significant TRPV1 inhibition effect compared to beta-sitosterol or betulin alone.

As used herein, the term “skin soothing” refers not only to alleviating and relieving erythema, itchy skin caused by irritation, or sensitive skin, etc., but also to the prevention of such symptoms, and may include alleviation of skin irritation. For example, the skin soothing may include, but is not limited to, relief of skin itching, alleviation of skin pain, and reduction of redness.

As used herein, the term “itching”, which is also referred to as pruritus, is defined as an unpleasant sensation that causes the desire to scratch the skin. In general, itching can occur independently of external stimuli, but can also be triggered by light mechanical contact with external substances, changes in ambient temperature, chemical substances, or electrical stimulation, etc. In the present disclosure, the “relieving itching” may be used interchangeably with the term anti-itch.

As used herein, the term “moisturization” refers to all activities of supplying moisture to the skin, blocking the evaporation of moisture to maintain the flexibility of the skin, and maintaining a smooth surface, and the term “dry” refers to a condition in which the skin is not sufficiently moisturized. Skin dryness is known to contribute to the breakdown of the skin barrier and triggers inflammation, and it is expected that an increase in skin moisturization may have a skin soothing effect in addition to a moisturizing effect.

In the present disclosure, hyaluronic acid can hold water equivalent to 300 to 1,000 times its own weight, and thus exhibits a moisturizing effect on the skin by combining with moisture.

In one embodiment of the present disclosure, it was confirmed that the composition of the present disclosure increased the expression of the hyaluronic acid synthesis gene (HAS3), thereby confirming an increase of moisture content in the skin and a moisturizing effect.

More specifically, the combination of beta-sitosterol and betulin of the present disclosure significantly increased HAS3 gene expression compared to beta-sitosterol or betulin alone.

Furthermore, the excellent moisturizing effect suggests that the composition of the present disclosure can prevent, improve, or alleviate skin diseases such as psoriasis, asteatosis, or eczema, etc.

As used herein, the term “anti-allergy” means to include improvement (alleviation of symptoms), treatment, and prevention (inhibition or delay of pathogenesis) of an allergic disease. An allergic disease refers to a pathological symptom caused by an allergic reaction mediated by mast cell activation such as mast cell degranulation, and the allergic disease may include. atopic dermatitis, asthma, allergic rhinitis, allergic conjunctivitis, allergic dermatitis, allergic contact dermatitis, and allergic keratitis, etc., but is not limited thereto.

The anti-allergy may suppress the secretion/expression of β-hexosaminidase in inflammatory cells by inhibiting degranulation of the inflammatory cells, or may suppress the secretion/expression of histamine, etc.

In one embodiment of the present disclosure, it was confirmed that the combination of beta-sitosterol and betulin; the combination of beta-sitosterol, betulin, and troxerutin exhibited excellent β-hexosaminidase secretion inhibition efficacy compared to beta-sitosterol, betulin, or troxerutin alone.

The composition including beta-sitosterol and betulin of the present disclosure may additionally comprise troxerutin.

As used herein, the term “troxerutin” refers to a hydroxyethyl ether derivative of rutin extracted from the pagoda tree, and is also known as vitamin P. It protects blood vessels, is effective in promoting capillary permeability and circulation, and prevents vascular fluid leakage, and thus is used for the treatment of hemorrhoids and varicose veins.

The troxerutin of the present disclosure may be those isolated or separated from an extract obtained from a plant containing troxerutin as an active ingredient, or a fraction thereof, but there is no limitation on the method for obtaining the same.

In the present disclosure, the troxerutin may be contained in an amount of 0.00001 to 3 wt % (w/w) based on the total weight of the composition. Specifically, it may be contained in an amount of 0.00001 to 3%, 0.001 to 2%, 0.01 to 1%, 0.05 to 1%, or 0.1 to 1%, but is not limited thereto.

Another aspect of the present disclosure provides a cosmetic composition for skin improvement, comprising beta-sitosterol and betulin as active ingredients.

The terms such as beta-sitosterol, betulin, skin improvement, etc., are as described in above.

The cosmetic composition may additionally comprise troxerutin.

In addition, the cosmetic composition may contain beta-sitosterol in an amount of 0.00001 to 3 wt % (w/w) based on the total weight of the composition, betulin in an amount of 0.00001 to 3 wt % (w/w) based on the total weight of the composition, and troxerutin in an amount of 0.00001 to 3 wt % (w/w) based on the total weight of the composition, but is not limited thereto.

In addition, the cosmetic composition may be used for improving skin permeation, but is not limited thereto.

In the present disclosure, it may be prepared in the form selected from the group consisting of solution, ointment for external use, cream, foam, nutritive cosmetic water, softening cosmetic water, facial mask, softening water, milky lotion, makeup base, essence, liquid washing agent, bath foam, sunscreen cream, sun oil, suspension, emulsion, paste, gel, lotion, powder, soap, surfactant-containing cleanser, oil, powder foundation, emulsion foundation, wax foundation, patch and spray, but is not limited thereto

The cosmetic composition of the present disclosure may further include one or more cosmetically acceptable carriers mixed with general skin cosmetics. As common ingredients, for example, oil, water, surfactants, moisturizers, lower alcohols, thickening agents, chelating agents, colorings, preservatives, fragrances, etc. may be appropriately mixed, but are not limited thereto.

The cosmetically acceptable carrier included in the cosmetic composition of the present disclosure may vary depending on the formulation of the cosmetic composition.

When the formulation of the present disclosure is an ointment, paste, cream or gel, animal oil, vegetable oil, wax, paraffin, starch, tragacanth, cellulose derivatives, polyethylene glycol, silicone, bentonite, silica, talc, zinc oxide, etc. may be used as a carrier ingredient, but the carrier ingredient is not limited thereto. These may be used alone or in a mixture of two or more thereof.

When the formulation of the present disclosure is a powder or spray, lactose, talc, silica, aluminum hydroxide, calcium silicate, polyamide powder, etc. may be used as a carrier ingredient, and in particular, when it is a spray, a propellant such as chlorofluorohydrocarbon, propane/butane or dimethyl ether may be additionally included, but the carrier ingredient is not limited thereto. These may be used alone or in a mixture of two or more thereof.

When the formulation of the present disclosure is a solution or emulsion, solvents, solubilizing agents or emulsifying agents, etc. may be used as a carrier ingredient, and for example, water, ethanol, isopropanol, ethyl carbonate, ethyl acetate, benzyl alcohol, benzyl benzoate, propylene glycol, 1,3-butyl glycol oil, cottonseed oil, peanut oil, maize germ oil, olive oil, castor oil, sesame seed oil, glycerol aliphatic ester, polyethylene glycol or aliphatic ester of sorbitan may be used, but the carrier ingredient is not limited thereto. These may be used alone or in a mixture of two or more thereof.

When the formulation of the present disclosure is a suspension, liquid diluents such as water, ethanol or propylene glycol, suspending agents such as ethoxylated isostearyl alcohol, polyoxyethylene sorbitol ester and polyoxyethylene sorbitan ester, microcrystalline cellulose, aluminum metahydroxide, bentonite, agar, or tragacanth, etc. may be used as a carrier ingredient, but the carrier ingredient is not limited thereto. These may be used alone or in a mixture of two or more thereof.

When the formulation of the present disclosure is a soap, alkali metal salts of fatty acids, salts of fatty acid hemiesters, fatty acid protein hydrolysates, isethionate, lanolin derivatives, aliphatic alcohol, vegetable oil, glycerol, sugars, etc. may be used as a carrier ingredient, but the carrier ingredient is not limited thereto. These may be used alone or in a mixture of two or more thereof.

Still another aspect of the present disclosure provides a quasi-drug composition comprising beta-sitosterol and betulin as active ingredients.

In the present disclosure, the composition may additionally comprise troxerutin.

The terms such as beta-sitosterol, betulin, skin improvement, etc., are as described in above.

In addition, the quasi-drug composition may contain beta-sitosterol in an amount of 0.00001 to 3 wt % (w/w) based on the total weight of the composition, betulin in an amount of 0.00001 to 3 wt % (w/w) based on the total weight of the composition, and troxerutin in an amount of 0.00001 to 3 wt % (w/w) based on the total weight of the composition, but is not limited thereto.

In addition, the quasi-drug composition may be used for improving skin permeation, but is not limited thereto

The quasi-drug composition of the present disclosure may further include a pharmaceutically acceptable carrier, excipient, or diluent, if necessary, in addition to the above ingredients. The pharmaceutically acceptable carrier, excipient, or diluent is not limited as long as it does not impair the effect of the present disclosure and may include, for example, a filler, an extender, a binder, a wetting agent, a disintegrant, a surfactant, a lubricant, a sweetener, an aromatic, a preservative, etc.

Examples of the quasi-drug composition of the present disclosure may include a disinfectant cleanser, a shower foam, an ointment, a wet wipe, a coating agent, etc., but is not limited thereto, and formulation methods, dosages, methods of use, constituting components, etc. of the quasi-drug can be appropriately selected from conventional techniques known in the art.

Yet another aspect of the present disclosure provides a method for skin improvement, including administering a composition comprising beta-sitosterol and betulin as active ingredients into a subject in need.

In the present disclosure, the composition may additionally comprise troxerutin.

The terms such as beta-sitosterol, betulin, skin improvement, etc., are as described above.

As used herein, the term “administration” means introducing the composition of the present disclosure into a patient by any appropriate method. An administration route of the composition may be any route, as long as it allows the composition to reach a target tissue. The composition of the present disclosure may be administered by applying on the skin, because it has a skin soothing effect or a skin irritation-relieving effect.

The composition of the present disclosure may be administered either daily or intermittently, and the administration frequency may be once or 2 to 3 times a day. Further, the composition of the present disclosure may be used alone or in combination with other drugs. Considering all the above factors, it is important to administer a minimum amount that may achieve a maximum effect without side effects, which may be readily determined by those skilled in the art.

As used herein, the term “subject” may refer to all animals, such as rats, mice, and livestock including humans, that has or may be likely to have skin irritation. Specifically, it may be a mammal including human.

Even another aspect of the present disclosure provides a method for improving neurogenic inflammation; a method for soothing skin; a method for relieving itching; a method for enhancing moisturization; a method for anti-allergy; or a method for relieving skin irritation, including administering a composition comprising beta-sitosterol and betulin as active ingredients into a subject in need.

In the present disclosure, the composition may additionally comprise troxerutin.

The terms such as beta-sitosterol, betulin, skin improvement, “improving neurogenic inflammation”, “skin soothing”, “relieving itching”, “enhancing moisturization”, “anti-allergy”, “relieving skin irritation”, etc., are as described above.

Further another aspect of the present disclosure to achieve the objects above provides a use for skin improvement; a use for improving neurogenic inflammation; a use for soothing skin; a use for relieving itching; a use for enhancing moisturization; a use for anti-allergy; or a use for relieving skin irritation of a composition comprising beta-sitosterol and betulin as active ingredients.

The composition including beta-sitosterol and betulin may additionally comprise troxerutin.

The terms such as beta-sitosterol, betulin, skin improvement, “improving neurogenic inflammation”, “skin soothing”, “relieving itching”, “enhancing moisturization”, “anti-allergy”, “relieving skin irritation”, etc., are as described above.

### Advantageous Effects

When the composition for skin improvement comprising beta-sitosterol and betulin as active ingredients according to the present disclosure was used, it was confirmed that the composition has excellent effects of skin-soothing and relieving skin irritation by reducing skin nerve stimulation-inducing factors, increasing the expression of moisturizing-related factors, increasing skin absorption and inhibiting mast cell activity, and thus can be effectively used in cosmetics, quasi-drugs, etc. for skin improvement.

### MODE FOR CARRYING OUT THE INVENTION

Hereinafter, the present disclosure will be described in more detail with reference to exemplary embodiments. However, the following exemplary embodiments are merely preferred embodiments for illustrating the present disclosure, and therefore, are not intended to limit the scope of the present disclosure thereto.

### Example 1. Evaluation of Inhibitory Effect of Transient Receptor Potential Vanilloid 1 (TRPV1) Activity (Cutaneous Neurogenic Irritation-Induced Receptor)

TRPV1-overexpressed HEK293 cells were suspended in DMEM medium, seeded into a 96-well plate, and cultured in a 37° C., CO₂ 5% incubator for 24 hours. Thereafter, the cells were washed with PBS buffer and treated with calcium binding dye using the Fluo-4 Direct™ Calcium Assay Kit from Invitrogen. Subsequently, the cells were treated with the test substances as shown in Table 1 below, and cultured in a 37° C., CO₂ 5% incubator for 40 minutes. To measure the degree of TRPV1 activation, 0.5 μM capsaicin was treated, and the fluorescence intensity was measured 2 minutes after treatment using a fluorescence microscope. In particular, the degree of TRPV1 activation (fluorescence) was calculated by the following formula using the background and peak point (Table 1).

$$Degree of TRPV 1 activation = \frac{{F}_{max} - F 0}{{F}_{0}}$$

Table 1 below shows the results of the decrease rate of TRPV1 activation, expressed as the inhibitory activity (%), when the cell lines prepared above were treated with each experimental substance compared to the untreated group, which was treated only with capsaicin.

TABLE 1

| | TRPV1 | Inhibitory |
|---|---|---|
| Experimental group | Activation | Activity [%] |
| Untreated group | 0.733692 | — |
| Sitosterol 2 ppm | 0.715268 | 2.5 |
| Sitosterol 4 ppm | 0.719871 | 1.9 |
| Sitosterol 10 ppm | 0.696333 | 5.1 |
| Betulin 10 ppm | 0.626015 | 14.7 |
| Betulin 20 ppm | 0.570974 | 22.2 |
| Betulin 10 ppm + Sitosterol 2 ppm | 0.604158 | 17.7 |
| Betulin 10 ppm + Sitosterol 10 ppm | 0.578603 | 21.1 |
| Betulin 20 ppm + Sitosterol 4 ppm | 0.532155 | 27.5 |
| Betulin 20 ppm + Sitosterol 10 ppm | 0.466911 | 36.4 |

As shown in the results in Table 1 above, the TRPV1 inhibitory activity of beta-sitosterol alone was not statistically significant from the baseline value. In contrast, when beta-sitosterol was combined with betulin, a significant TRPV1 inhibitory activity was observed.

Through these results, it was confirmed that beta-sitosterol and betulin of the present disclosure exhibited excellent effects in improving neurogenic inflammation, reducing neurogenic irritation/pain, skin soothing, relieving itching, and improving skin conditions, etc.

### Example 2. Evaluation of Effect of Increasing the Activity of HAS3 (Hyaluronic Acid Synthase 3), a Marker of Skin Moisturization

Keratinocytes were suspended in a 0.01 mM Ca²⁺ DMEM medium, seeded into a 12-well plate, and cultured in a 37° C., CO₂ 5% incubator for 24 hours. The cultured cells were washed with PBS and treated with the test substances as listed in Table 2. Thereafter, RNA was extracted from the cells using an RNA extraction kit (AccuPrep® Universal RNA Extraction Kit), and RT-qPCR was performed using TOPscript™ RT DryMIX (dN6) and TOPreal™ SYBR Green RT-qPCR Kit to measure the HAS3 gene expression level (Table 2).

Table 2 below shows the relative values (percentage %) of HAS3 expression levels when treated with each test substance compared to the untreated group.

TABLE 2

| | Increase in Expression |
|---|---|
| Experimental group | level of HAS3 |
| Sitosterol 0.4 ppm | 19.47 |
| Sitosterol 2 ppm | −6.04 |
| Betulin 0.4 ppm | 4.72 |
| Betulin 2 ppm | 34.41 |
| Betulin 0.4 ppm + Sitosterol 2 ppm | 25.12 |
| Betulin 2 ppm + Sitosterol 0.4 ppm | 94.98 |
| Betulin 2 ppm + Sitosterol 2 ppm | 44.72 |

As shown in the results in Table 2 above, it was confirmed that the combination of beta-sitosterol and betulin significantly increased HAS3 gene expression levels compared to beta-sitosterol or betulin alone.

These results indicate that the beta-sitosterol and betulin of the present disclosure exhibit an excellent skin moisturization effect by increasing skin moisture content.

### Example 3. Evaluation of Skin Permeability

To determine the skin permeation efficiency of the active ingredients, skin permeability was evaluated using cream compositions comprising beta-sitosterol or betulin. The cream compositions contained the following ingredients in both the oil phase and the water phase, respectively:

### <Cream Composition>

Oil-phase: cetyl stearyl alcohol, glyceryl stearate, PEG-40 stearate, ceteareth-20, beeswax, C14-22 alcohols, C12-20 alkyl glucoside, lecithin, tocopherol, caprylic/capric triglyceride, squalene, cyclopentasiloxane, cyclohexasiloxane, dimethicone/vinyl dimethicone crosspolymer, isocetyl myristate

Water-phase: dipropylene glycol, glycerin, betaine, 1,2-hexanediol, EDTA-3Na, xanthan gum, carbomer, tromethamine, water.

All oil-phase ingredients were placed in a beaker and slowly heated to 60° C. while stirring. All water-phase ingredients were placed in another beaker and slowly heated to 60° C. while stirring. Thereafter, the water phase was gradually added to the oil phase while stirring to emulsify the mixture. Once emulsification was complete, the temperature was allowed to cool to room temperature.

In the above process, betulin was added to the oil phase at a concentration of 0.1% based on the mass of the final composition. Sitosterol was also added to the oil phase of each cream composition at concentrations shown in Table 3 based on the mass of the final composition.

After applying 100 μl of the prepared cream to the upper part (stratum corneum side) of pig skin (Apures, 2.5 cm×2.5 cm), the pig skin was fixed onto the Franz diffusion cell equipment and maintained in a 37° C. incubator for 24 hours. An ethanol/PBS (1:1) solution was used as a reservoir solution. Thereafter, the pig skin was pulverized with a homogenizer, and 1 ml of ethanol/PBS (1:1) was added to sufficiently dissolve the betulin, and then the mixture was centrifuged at 12,000 rpm for 10 minutes. After centrifugation, the supernatant was collected, and the total mass of betulin contained in each of the supernatant and the reservoir was quantified, and these were added to obtain the total permeation amount of betulin. The quantification of betulin was performed by RP-HPLC, and the conditions are as follows.
mobile phase: acetonitrile-water (86:14, v/v), Isocratic
Injection volume 20 μl, flow rate 1 ml/min
Absorbance measured at 210 nm

TABLE 3

| | sitosterol 0% | sitosterol 0.01% | sitosterol 0.1% |
|---|---|---|---|
| Permeation amount | 3.62 | 4.25 | 10.71 |
| of betulin (μg) | | | |

As shown in the results in Table 3 above, the combination of beta-sitosterol and betulin increased skin permeability compared to betulin alone.

Accordingly, it was found that the beta-sitosterol and betulin of the present disclosure increased skin permeability, demonstrating excellent skin improvement effects.

### Example 4. Evaluation of Stability

Betulin and sitosterol have low solubility in various solvents, including water, which can easily lead to problems such as precipitation and stability. In this experiment, the effect of combining the two poorly soluble components was examined on their solubilities.

First, in order to simulate a composition containing a large amount of organic solvent and water, a solvent was prepared by mixing DPG and distilled water in a 3:4 ratio. In order to facilitate the experiment, poloxamer 188, a solubilizer, was added to the solvent at 1% weight ratio. Then, the test substances and the prepared solvent were placed in 20 mL glass vials at the specified weight ratios, as shown in Table 4. In particular, the final composition contained in each vial was 10 g. Subsequently, the glass vials were stirred for 30 minutes and allowed to stand for 1 minute, and observed for precipitation. The results are shown in Table 4.

TABLE 4

| Experimental group | Precipitation |
|---|---|
| Betulin 0.014% | Occurred |
| Sitosterol 0.014% | Occurred |
| Betulin 0.014% + Sitosterol 0.014% | Not occurred |

As a result, it was confirmed that a precipitate was formed when beta-sitosterol and betulin were used alone, while no precipitate was formed when beta-sitosterol and betulin were used in combination.

Accordingly, it was confirmed that in the case of compositions containing a large amount of organic solvent or water, the solubility was improved when the components were used in combination compared to when each component was used alone.

### Example 5. Evaluation of Inhibitory Effect on Mast Cell Activation

Mast cells (RBL-2H3) were suspended in DMEM culture medium, seeded into a 24-well plate, and cultured in a 37° C., CO₂ 5% incubator for 15 hours. The cells were treated with the test substances according to Tables 5 to 7 for 2 hours. Thereafter, the cells were washed with Siraganian buffer, reacted with C48/80 (50 μg/ml in Siraganian buffer) as a stimulus for 30 minutes, and the reaction was terminated by placing the culture plate on ice. Herein, 50 μl of the reaction supernatant was collected and transferred to a 96-well plate, and 50 μl of substrate buffer (2 mM 4-Nitrophenyl N-acetyl-β-D-glucosaminide, 0.05 M sodium citrate, pH 4.5) was added, and the mixture was reacted for 90 minutes. Then, 200 μl of stop buffer (0.1 M sodium carbonate/sodium bicarbonate, pH 10.5) was added to terminate the reaction. Subsequently, β-Hexosaminidase, a degranulation indicator, was quantified by measuring absorbance at 405 nm. The amount of β-hexosaminidase released was calculated as a relative value to the amount released from the untreated group (negative control group). The β-hexosaminidase release inhibition rate was calculated as a reduced amount of β-hexosaminidase released when the test substance was treated (experimental group) relative to the amount released from the positive control group treated only with C48/80 (50 μg/ml in Siraganian buffer), which was not treated with the test substance, which was expressed as the percentage (%), as shown in Tables 5 to 7.

$$β - Hexosaminidase release inhibition rate = ( positive control group - experimental group ) \div positive control group \times 100$$

TABLE 5

| | Degree of |
|---|---|
| Test substance | inhibition (%) |
| sitosterol 6.25 ppm | 3.99 |
| betulin 6.25 ppm | 2.17 |
| sitosterol 6.25 ppm + betulin 6.25 ppm | 7.87 |
| troxerutin 6.25 ppm | 4.79 |
| sitosterol 6.25 ppm + betulin 6.25 ppm + | 14.77 |
| troxerutin 6.25 ppm | |

TABLE 6

| | Degree of |
|---|---|
| Test substance | inhibition (%) |
| sitosterol 12.5 ppm | 11.08 |
| betulin 12.5 ppm | 8.75 |
| sitosterol 12.5 ppm + betulin 12.5 ppm | 23.15 |
| troxerutin 12.5 ppm | 12.90 |
| sitosterol 12.5 ppm + betulin 12.5 ppm + | 44.88 |
| troxerutin 12.5 ppm | |

TABLE 7

| | Degree of |
|---|---|
| Test substance | inhibition (%) |
| sitosterol 6.25 ppm | 3.99 |
| betulin 12.5 ppm | 8.75 |
| sitosterol 6.25 ppm + betulin 12.5 ppm | 13.08 |
| troxerutin 6.25 ppm | 4.79 |
| sitosterol 6.25 ppm + betulin 12.5 ppm + | 24.55 |
| troxerutin 6.25 ppm | |
| troxerutin 12.5 ppm | 12.90 |
| sitosterol 6.25 ppm + betulin 12.5 ppm + | 36.38 |
| troxerutin 12.5 ppm | |
| troxerutin 50 ppm | 26.45 |
| sitosterol 6.25 ppm + betulin 12.5 ppm + | 48.12 |
| troxerutin 50 ppm | |
| troxerutin 100 ppm | 26.45 |
| sitosterol 6.25 ppm + betulin 12.5 ppm + | 48.54 |
| troxerutin 100 ppm | |

As shown in Tables 5 to 7 above, the combination of beta-sitosterol and betulin exhibited an excellent inhibitory effect of β-hexosaminidase secretion compared to beta-sitosterol and betulin alone. In addition, the combination of beta-sitosterol, betulin, and troxerutin demonstrated a significantly greater inhibitory effect of β-hexosaminidase secretion compared to beta-sitosterol, betulin, or troxerutin alone.

As a result, the combination of beta-sitosterol and betulin, and the combination of beta-sitosterol, betulin, and troxerutin, effectively inhibited mast cell degranulation, demonstrating excellent anti-allergy, anti-itch, skin soothing, and skin irritation relief effects.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the present disclosure is defined by the appended claims rather than by the description preceding them, and therefore all changes and modifications that fall within metes and bounds of the claims, or equivalents of such metes and bounds, are therefore intended to be embraced by the claims.

1. A method of improving skin in a subject in need thereof, the method comprising administering an affective amount of a composition comprising beta-sitosterol and betulin as active ingredients, to the subject.

2. The method according to claim 1, wherein the beta-sitosterol is contained in an amount of 0.00001 to 3 wt % (w/w) based on the total weight of the composition.

3. The method according to claim 1, wherein the betulin is contained in an amount of 0.00001 to 3 wt % (w/w) based on the total weight of the composition.

4. The method according to claim 1, wherein the skin improvement is any one selected from the group consisting of improving neurogenic inflammation, skin-soothing, relieving itching, enhancing moisturization, anti-allergy, and relieving skin irritation.

5. The method according to claim 1, wherein the composition further comprises troxerutin.

6. The method according to claim 5, wherein the troxerutin is contained in an amount of 0.00001 to 3 wt % (w/w) based on the total weight of the composition.

7. The method according to claim 1, wherein the composition is a cosmetic composition.

8. The method according to claim 1, wherein the composition is a quasi-drug composition.
